(19) United States
(12) Patent Application Publication
(10) Pub. No.: US 2025/0152892 A1
(43) Pub. Date: May 15, 2025

(54) NASAL CANNULA WITH HME

(71) Applicant: AEON RESEARCH AND TECHNOLOGY, INC., Hewlett, NY (US)

(72) Inventors: Sunil Kumar DHUPER, Old Westbury, NY (US); Greg MARLER, Rockford, IL (US)

(73) Assignee: AEON RESEARCH AND TECHNOLOGY, INC., Hewlett, NY (US)

(21) Appl. No.: 18/865,514

(22) PCT Filed: May 12, 2023
(86) PCT No.: PCT/US2023/022080

§ 371 Date: Nov. 13, 2024

Related U.S. Application Data
(60) Provisional application No. 63/341,808, filed on May 13, 2022.

(51) Int. Cl. A61M 16/06; A61M 16/10

(52) U.S. Cl. CPC A61M 16/0672; A61M 16/1045

(57) ABSTRACT

A nasal cannula includes a housing having a first end and an opposite second end. The nasal cannula also includes a pair of supplemental gas inlets that are in fluid communication with an interior of the housing and extend outwardly from the housing. A pair of nasal ports are in fluid communication with the interior of the housing and extend outwardly from the housing. The nasal cannula also include one or more HMEs that are disposed within the interior of the housing and positioned such that supplemental gas flowing through the pair of supplemental gas inlets into the housing flows through the one or more HMEs to reach the pair of nasal ports.

### CROSS REFERENCE TO RELATED APPLICATION

The present disclosure claims priority to and the benefit of U.S. patent application Ser. No. 63/341,808, filed May 13, 2022, which is hereby incorporated by reference in its entirety.

### TECHNICAL FIELD

The present application is directed to nasal cannulas and more particularly, is directed to an improved nasal cannula with a heat moisture exchanger (HME).

### BACKGROUND

A traditional nasal cannula is a device that is used to deliver supplemental oxygen or increased airflow to a patient or person in need of respiratory help. The device consists of a lightweight tube which on one end splits into two prongs which are placed in the nostrils and from which a mixture of air and oxygen flows. The two prongs loosely fit in the nostrils and thus, air can enter around the two prongs into the nostrils in addition to oxygen delivered by the nasal cannula. These two prongs must fit loosely in the nostrils since the patient must exhale out of the nostrils since the prongs are part of a closed system and the only means for the patient to exhale is by exhaling around the prongs through the nostrils. The other ends of the tube are connected to an oxygen supply, such as a portable oxygen generator, or a wall connection in a hospital via a flowmeter. The nasal cannula is typically attached to the patient by way of the tube hooking around the patient's ears or by an elastic headband. A traditional flow rate is 1-6 liters of oxygen per minute that is delivered to the patient.

One of the challenges of using a nasal cannula is that the gas delivered is dry and can result in the nasal passages and sinuses of the patient becoming very dry with discomfort, crust formation, bleeding and susceptible to infections which may even require antibiotics.

### SUMMARY

A nasal cannula includes a housing having a first end and an opposite second end. The nasal cannula also includes a pair of supplemental gas inlets that are in fluid communication with an interior of the housing and extend outwardly from the housing. A pair of nasal ports are in fluid communication with the interior of the housing and extend outwardly from the housing. The nasal cannula also includes one or more HMEs that are disposed within the interior of the housing and positioned such that supplemental gas flowing through the pair of supplemental gas inlets into the housing flows through the HME(s) to reach the pair of nasal ports.

Unlike the completely closed system of the prior art, the nasal cannula of the present disclosure has fluid communication to atmosphere through the housing itself. In particular, the nasal ports (prongs) are flexible and oversized so that they occupy the nostrils. In other words, the nasal ports fill the nostrils and seat against the nostril walls so as to prevent air from flowing around the nasal ports both during inhalation and especially during exhalation. As a result, during exhalation, exhaled air travels back through the nasal ports, through the HME and then exits through the ends of the housing which are open to atmosphere. Since exhaled air must pass through the HME during exhalation (unlike conventional design in which exhaled air exits through the nostrils around the nasal ports to atmosphere), the patient's own humidity is used to humidify the inhaled air. The patient's humidity is thus used as opposed to conventional design in which external humidity is used.

### BRIEF DESCRIPTION OF THE DRAWING FIGURES

FIG. 1 is a front perspective view of an HME nasal cannula coupled to tubing;

FIG. 2 is a perspective view of the HME nasal cannula in an exploded view of a housing thereof being in an open position;

FIG. 3 is a perspective view of the HME installed in the open housing;

FIG. 4 is a perspective view of the nasal cannula in a closed position;

FIG. 5 is a cross-sectional view of the HME nasal cannula;

FIG. 6 is a top plan view of an HME nasal cannula according to another embodiment; and

FIG. 7 is a cross-sectional view of the HME nasal cannula of FIG. 6.

### DETAILED DESCRIPTION OF CERTAIN EMBODIMENTS

### Nasal Cannula Housing

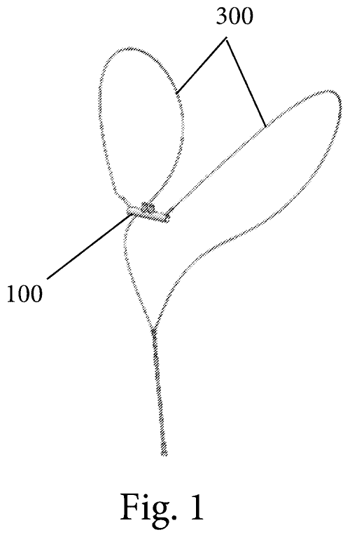

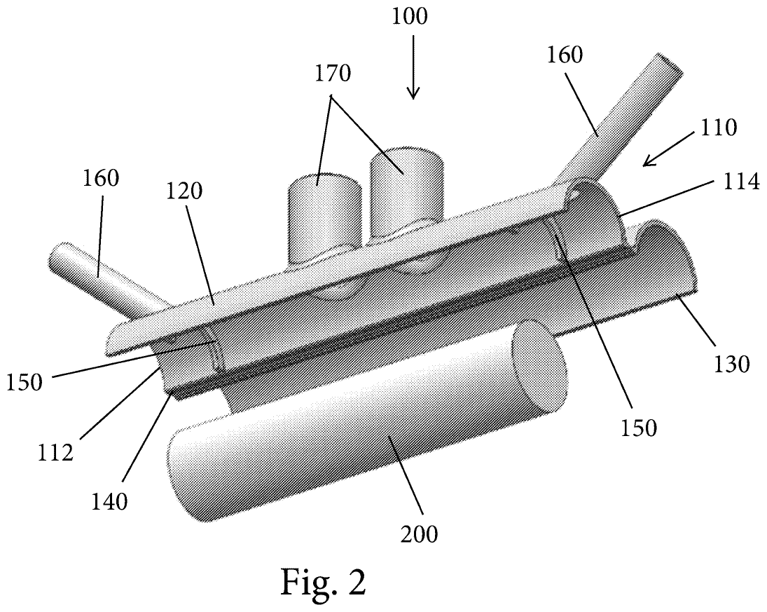

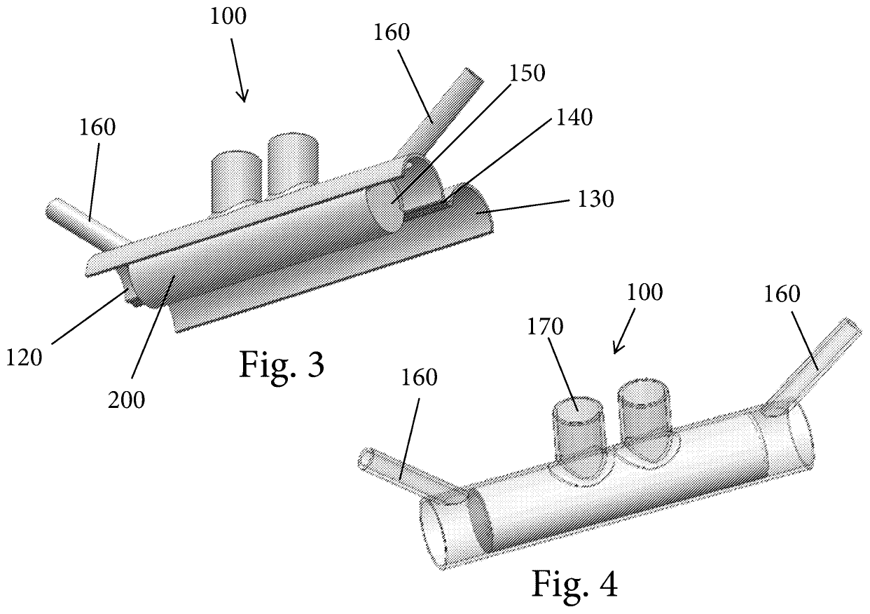

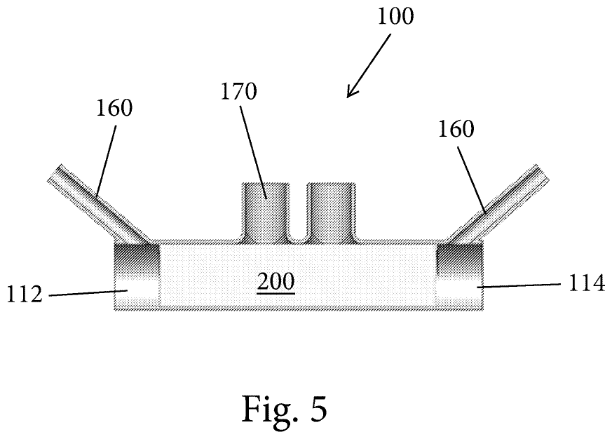

FIGS. 1-5 illustrate a nasal cannula 100 in accordance with one embodiment of the present disclosure. The nasal cannula 100 has a housing 110 that can be formed of a first part 120 and a second part 130 that are coupled to one another. The housing 110 has a first end 112 and an opposite second end 114. As illustrated, the first end 112 and the second end 114 are open ends. These open ends 112, 114 provide atmospheric air flow in both directions in that atmospheric air can be inhaled air and the open ends 112, 114 define exhalation exit ports through which the exhaled air exits after flowing through one or more HMEs.

Each of the first part 120 and the second part 130 can have a truncated cylinder or half cylinder shape as shown. The first part 120 can be thought of as being a top part and the second part 130 can be thought of as a bottom part. The first part 120 can be coupled to the second part 130 by a living hinge 140 or the like that permits the first part 120 and second part 130 to be moved between an open position and a closed position. In the closed position, the first part 120 and the second part 130 form a cylinder that is open at the first end 112 and the second end 114. The illustrated embodiment can thus be considered to be of a clamshell design with the living hinge 140 connecting the two parts 120, 130 of the clamshell.

It will be appreciated that any number of locking mechanisms can be used to securely attach the first part 120 to the second part 130. For example, a latch assembly can be used (e.g., a releasable snap-fit) to lock the first part 120 to the second part 130 in the closed position.

Inside of at least one of the first part 120 and the second part 130 is a single retention tab or a pair or plurality of retention tabs (retainers) 150. Each retention tab 150 can be in the form of a semi-circular flange that protrudes outwardly from the inner surface of the first part 120 and/or second part 130. The one or more retention tabs 150 can be integrally formed within the respective first part 120 or second part 130 (i.e., can be a single molded structure). In the illustrated embodiment, the two retention tabs 150 are formed as part of the first part 120.

It will be appreciated that the housing 110 is not limited to having a clamshell design and other constructions are equally possible. For example, the housing 110 can be of a single body construction. For example, the housing 110 can be a hollow structure that is open at at least one end thereof. For example, the housing 110 can be a hollow cylinder that is closed at one end and open at the other end or in another embodiment, the housing can be a hollow cylinder open at both ends. In this design, the housing 110 is of a one piece construction.

### Supplemental Gas Inlets

The housing 110 includes at least one supplemental gas inlet 160 and can be a pair of supplemental gas inlets 160 that are offset from the ends 112, 114 of the housing 110. The supplemental gas inlets 160 are associated with one of the first part 120 and the second part 130. In the illustrated embodiment, the supplemental gas inlets 160 are associated with the first part 120. As illustrated, the supplemental gas inlets 160 are configured to be fluid coupled to the source of supplemental gas (e.g., oxygen) at first ends thereof with the opposite second ends being in fluid communication with the inside of the housing 110 so as to deliver the supplemental gas into the inside of the housing 110. In the illustrated embodiment, the second ends of the supplemental gas inlets 160 are located between the retention tabs 150 with the ends 112, 114 but could also be located anywhere between the retention tabs 150 and nasal ports 170. The supplemental gas inlets 160 can be formed at an angle relative to the housing 110, such as an angle other than a 90 degree angle. As illustrated, the supplemental gas inlets 160 can be formed to direct the supplemental gas that flows within the inlets 160 toward the center of the housing 110.

The supplemental gas inlets 160 are thus tubular structures that can be integrally formed within the housing 110. As mentioned, in the illustrated embodiment, the supplemental gas inlets 160 are integrally formed with the first part 120.

It will also be appreciated that the one or more supplemental gas inlets 160 can be positioned at different positions along the housing 110 than shown in the figures.

### Nasal Ports

The housing 110 also includes a pair of nasal ports 170 that extend outwardly from the housing 110 and are intended for insertion into the nostrils of the patient.

In accordance with the present disclosure, the pair of nasal ports 170 are intended to occupy an at least substantial area of the nostrils (near complete or complete occupancy of the nostrils). The nasal ports 170 thus are oversized and seat against the inside of the nostrils to seal the nostrils. For example, the pair of nasal ports 170 can be sized and formed of a flexible material such that the pair of nasal ports 170 contact the inside of the nostrils 170. The pair of nasal ports 170 can be formed of a soft flexible (pliable) elastomeric material (e.g., a silicone material) that can conform to the inside of the nostrils. In contrast to the loose fit of the traditional nasal ports within the nostrils, the pair of nasal ports 170 fit snuggly with the nostrils and thus prevents leakage around the nasal ports 170. This occupancy of the nasal ports 170 in the nostrils prevents exhaled air from existing the nostrils around the nasal ports 170 and instead, the exhaled air must flow through the nasal ports 170 into the housing and into contact with the HME prior to exiting through open ends 112 and 114. Similarly, the inhaled air does not flow around the nasal ports 170 but instead passes internally within the nasal ports 170.

As shown, the pair of nasal ports 170 are formed relatively parallel or at an acute angle to one another and are formed at a 90 degree angle to the housing 110. In the illustrated embodiment, the pair of nasal ports 170 are formed as part of the first part 120. The pair of nasal ports 170 are located between the supplemental gas inlets 160.

In the case of one single supplemental gas inlet 160, the supplemental gas inlet 160 is not positioned between the two nasal ports 170.

In the event that the housing 110 is of a single piece construction, the two nasal ports 170 are located along one surface (e.g., one side) of the housing 110.

### Heat Moisture Exchanger

A heat and moisture exchanger (HME) 200 is provided and is disposed within the housing 110 and is within the supplemental gas flow pathway through the housing 110. More particularly, devices in accordance with the present disclosure are constructed such that the inhaled air, which is made up of the supplemental gas and ambient air, flows through the HME to the nasal ports 170 and exhaled air flows through the HME to an exhalation port.

As is known, a heat and moisture exchanger is a device that is used in mechanically ventilated patients or spontaneously breathing patients intended to help prevent complications due to drying of the respiratory mucosa, such as mucus plugging and endotracheal tube (ETT) occlusion. The HME 200 is typically formed of hygroscopic foam or paper treated with a salt solution, such as calcium chloride.

In the illustrated embodiment, the HME 200 has a cylindrical shape and is sized and shaped to fit within the interior of the housing 110. The HME 200 is held by and between the retention tabs 150. For example, the HME 200 can have a length that is greater than a distance between the retention tabs 150; however, the HME 200 is compressible and therefore, it can be fit and disposed between the retention tabs 150. The HME 200 thus fits snugly between the retention tabs 150 which serve to hold the HME 200 in place. The HME 200 can be disposed between the retention tabs 150 and can thus be held in place within the first part 120 when the housing 110 is open. In this arrangement, half of the HME 200 is contained within the first part 120, while the other half lies outside the first part. When the first and second parts 120, 130 close, the other half enters into and is contained within the second part 130.

It will be appreciated that the majority volume of the HME 200 lies between the supplemental gas inlets 160 and the pair of nasal ports 170 are located adjacent the HME 200. As a result, the supplemental gas that flows through the supplemental gas inlets 160 is delivered to locations adjacent the opposite ends of the HME 200 and flows through the HME 200 to reach the nasal ports 170 and then ultimately flow into the nostrils of the patient.

The open ends 112, 114 of the housing 110 permit atmospheric air to flow into the housing 110 and more particularly, flow through the HME 200 to the nasal ports 170. This arrangement allows for mixing of atmospheric air and the supplemental gas at locations immediately adjacent the ends of the HME 200.

It will also be appreciated that any exhaled air will also pass through the HME 200 since the exhaled air exits through the nasal ports 170 into the HME 200 and then exits at the ends 112, 114 of the housing 110.

The clamshell nature of the housing 110 allows the HME 200 to be easily replaced by simply opening up the first and second parts 120, 130 and removing the HME 200.

In alternative constructions when the housing 110 has a single piece construction, the HME 200 is disposed within the hollow interior thereof. For example, when the housing 110 is a hollow cylinder, the cylinder can be closed at one end and the HME 200 is inserted therein. A single supplemental gas inlet is located outside the HME 200, such as at the open end of the cylinder. An exhalation port (outlet) is also located at the open end. As in the other constructions, the inhaled supplemental gas and ambient air flow through the HME 200 into the nasal ports 170 to the patient. During exhalation, the exhaled gas flows through the HME 200 to the exhalation outlet.

In addition, as described below, the HME can be formed of two HMEs or more.

### Tubing

As in a traditional setup, tubing 300 is used to connect the nasal cannula 100 to the oxygen source (not shown) as shown in FIG. 1.

### Gas Flow Path

In all of the embodiments, the housing includes the pair of nasal ports 170 and at least one supplemental gas inlet and at least one exhalation port (outlet). The gas flow path is such that supplemental gas and ambient air is pulled through the HME 200 by an inhalation action of the patient and then enters into and passes through the nasal ports 170 to the patient. When the patient exhales, the exhaled air flows back through the HME to one or more exhalation ports. Each supplemental gas inlet is not formed directly over the HME to allow supplemental gas to flow into two mixing chambers.

### Multiple HMEs

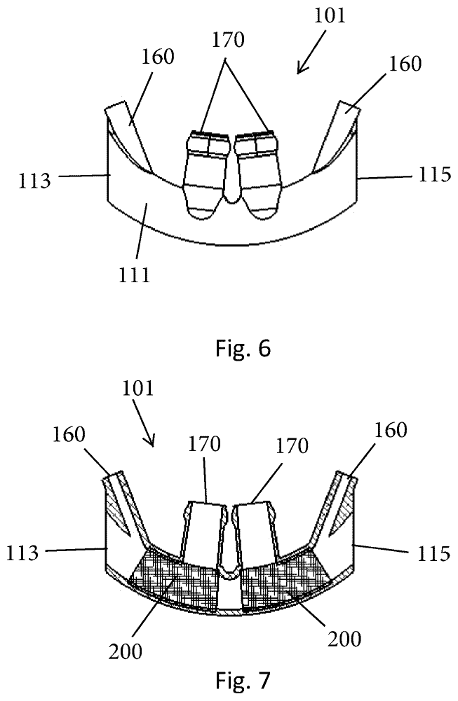

Now referring to FIGS. 6 and 7 in which a nasal cannula 101 in accordance with one embodiment of the present disclosure is illustrated. The nasal cannula 101 is similar to the nasal cannula 100.

The nasal cannula 101 includes a housing 111 that has a hollow interior and has a first end 113 and an opposite second end 115. The first and second ends 113, 115 are open and permit both inhalation and exhalation gas flow. The housing 111 can be curved as shown and thus, the hollow interior can have an arcuate shape.

The housing 111 includes at least one supplemental gas inlet 160 and there can be a pair of supplemental gas inlets 160 that are offset from the ends 113, 115 of the housing 111. As shown, the supplemental gas inlets 160 can be angled and protrude outwardly from the housing 111. The supplemental gas inlets 160 have exposed ends to which gas tubing can be attached to deliver the supplemental gas from a source (reservoir/tank). The supplemental gas inlets 160 are thus in fluid communication with the hollow interior and thus supplemental gas can be delivered through the supplemental gas inlets 160 and can mix with air entering through ends 113, 115.

The housing 111 also includes a pair of the nasal ports 170 that extend outwardly from the housing 111 and are intended for insertion into the nostrils of the patient. The nasal ports 170 are located between the supplemental gas inlets 160 and thus are spaced inwardly from the ends 113, 115.

In the nasal cannula 101, there are two HMEs 200. One HME 200 is positioned at the location of one nasal port 170 and one HME 200 is positioned at the location of the other nasal port 170. The HMEs 200 are inserted into the hollow interior of the housing 111. The inner ends of the two HMEs 200 can be spaced apart (as shown) or they can be in contact. The HMEs 200 are removable from the housing 111. The outer ends of the HMEs 200 do not cover or obstruct the supplemental gas inlets 160 as shown. Thus, the areas of the housing 111 between the outer ends of the HMEs 200 and the respective ends 113, 115 are open spaces that communicate with both the supplemental gas inlets 160 and the open ends 113, 115 to allow both a mixing of gases for inhalation and permit exhaled gas to exit through ends 113, 115 (which function as exhalation ports/outlets).

As in the other embodiment, the HMEs 200 are within the supplemental gas flow pathway through the housing 111. More particularly, devices in accordance with the present disclosure are constructed such that the inhaled air, which is made up of the supplemental gas and ambient air, flows through the HME to the nasal ports 170 and exhaled air flows through the HMEs 200 to exhalation ports.

It is to be understood that like numerals in the drawings represent like elements through the several figures, and that not all components and/or steps described and illustrated with reference to the figures are required for all embodiments or arrangements.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

The subject matter described above is provided by way of illustration only and should not be construed as limiting. Various modifications and changes can be made to the subject matter described herein without following the example embodiments and applications illustrated and described, and without departing from the true spirit and scope of the present invention, which is set forth in the following claims.

What is claimed is:

1. A nasal cannula comprising:
a housing having a first end and an opposite second end;
at least one supplemental gas inlet that is in fluid communication with an interior of the housing and extends outwardly from the housing for attachment to a source of supplemental gas;
a pair of nasal ports that are in fluid communication with the interior of the housing and extend outwardly from the housing; and
an HME that is disposed within the interior of the housing and positioned such that supplemental gas flowing through the at least one supplemental gas inlet into the housing flows through the HME to reach the pair of nasal ports.

2. The nasal cannula of claim 1, wherein the at least one supplemental gas inlet comprises a pair of supplemental gas inlets each of which is spaced from the HME.

3. The nasal cannula of claim 2, wherein the first and second ends of the housing are open and the housing has a first mixing chamber defined between one outer end of the HME and the first end and a second mixing chamber defined between another outer end of the HME and the second end with one supplemental gas inlet opening into the first mixing chamber and the other supplemental gas inlet opening into the second mixing chamber, the open first and second ends defining exhalation outlets.

4. The nasal cannula of claim 2, wherein the HME comprises a pair of HMEs disposed within the interior with one HME disposed against and in fluid communication with one supplemental gas inlet and the other HME disposed against and in fluid communication with the other supplemental gas inlet.

5. The nasal cannula of claim 1, wherein the housing comprises a first part and a second part that are configured such that the housing can move between an open position and a closed position.

6. The nasal cannula of claim 2, wherein the pair of supplemental gas inlets are tubular structures that are formed at an angle other than 90 degrees relative to the housing.

7. The nasal cannula of claim 2, wherein the pair of nasal ports are disposed between the pair of supplemental gas inlets.

8. The nasal cannula of claim 1, wherein the pair of nasal ports are formed of a flexible compliant material to allow for seating against inner walls of the nostrils.

9. The nasal cannula of claim 1, wherein the housing includes one or more retention tabs against which one or more HMEs seat to allow the one or more HMEs to be held in place within the housing between the one or more retention tabs.

10. The nasal cannula of claim 9, wherein each retention tab has a semi-circular shape or arcuate shape.

11. The nasal cannula of claim 10, wherein the at least one supplemental gas inlet comprises a pair of supplemental gas inlets each of which is spaced from the HME and wherein the housing comprises a first part and a second part that are configured such that the housing can move between an open position and a closed position and the retention tabs are formed along an inner face of the first part, wherein the pair of nasal ports and the pair of supplemental gas inlets are formed as part of the first part.

12. The nasal cannula of claim 11, wherein the pair of supplemental gas inlets open into the first part at locations between the nasal ports and open ends of the first part.

13. The nasal cannula of claim 1, wherein the HME has a cylindrical shape and is held within the interior of the housing by a friction fit between a pair of retention tabs.

14. The nasal cannula of claim 13, wherein the housing comprises a first part and a second part that are configured such that the housing can move between an open position and a closed position and the retention tabs are formed along an inner face of the first part and the HME is held within the first part.

15. The nasal cannula of claim 1, wherein a center axis of each of the supplemental gas inlets intersects the HME or a respective end of the HME and the supplemental gas inlets are connected to the housing at a location allowing incoming gas to pass through the HME prior to exiting through the nasal ports.

16. The nasal cannular of claim 1, wherein the first and second ends of the housing include mixing chambers that defined between ends of the HME and the respective first and second ends of the housing, which is the pair of supplemental gas inlets are in direct fluid communication with the mixing chambers and the first and second ends of the housing are open to atmosphere.

17. The nasal cannula of claim 1, wherein the pair of nasal ports are formed of a material that is different than a material of the housing.

18. A nasal cannula comprising:
a housing having an open first end and an opposite open second end;
a pair of supplemental gas inlets that are in fluid communication with an interior of the housing, each supplemental gas inlet extending outwardly from the housing for attachment to a source of supplemental gas;
a pair of nasal ports that are in fluid communication with the interior of the housing and extend outwardly from the housing; and
first and second HMEs that are disposed within the interior of the housing and positioned such that supplemental gas flowing through one supplemental gas inlet into the housing flows through the first HME to reach one nasal port and supplemental gas flowing through the other supplemental gas inlet into the housing flows through the second HME to reach the other nasal port.

19. The nasal cannula of claim 1, wherein the housing has a first mixing chamber defined between and outer end of the first HME and the first end and a second mixing chamber defined between an outer end of the second HME and the second end with the one supplemental gas inlet opening into the first mixing chamber and the other supplemental gas inlet opening into the second mixing chamber, the open first and second ends defining exhalation outlets.

20. The nasal cannula of claim 19, wherein the one supplemental gas inlet is adjacent the outer end of the first HME and the one nasal port is at an inner end of the first HME and the other supplemental gas inlet is adjacent the outer end of the second HME and the other nasal port is at an inner end of the second HME.
